(19) United States
(12) Patent Application Publication
(10) Pub. No.: US 2023/0238085 A1
(43) Pub. Date: Jul. 27, 2023

(54) METHOD AND APPARATUS FOR DETERMINING MOLECULAR CONFORMATION

(71) Applicants: SAMSUNG ELECTRONICS CO., LTD., Suwon-si (KR); La Corporation de l’École des Hautes Études Commerciales de Montréal, Montreal (CA)

(72) Inventors: MiYoung Jang, Hanam-si (KR); HWIDONG NA, Seongnam-si (KR); Jian Tang, Montreal (CA); SANGHYUN YOO, Seoul (KR); HOSHIK LEE, Seongnam-si (KR)

(73) Assignees: SAMSUNG ELECTRONICS CO., LTD., Suwon-si (KR); La Corporation de l'École des Hautes Études Commerciales de Montréal, Montreal (KR)

(21) Appl. No.: 17/868,417

(22) Filed: Jul. 19, 2022

(30) Foreign Application Priority Data
Jan. 24, 2022 (KR) 10-2022-0009779

(51) Int. Cl. G16C 20/30; G16C 20/10; G16C 20/70

(52) U.S. Cl. CPC G16C 20/30; G16C 20/10; G16C 20/70

(57) ABSTRACT

Provided is a method and apparatus for determining a molecular conformation. The method of determining a molecular conformation includes generating candidate conformations based on one or more artificial neural network (ANN)-based conformation generative model that is based on an artificial neural network (ANN), comparing energy values between the candidate conformations by inputting the candidate conformations to an ANN-based conformation selecting model, and determining a final conformation based on a result of the comparing.

### CROSS-REFERENCE TO RELATED APPLICATIONS

This application claims the benefit under 35 USC § 119(a) of Korean Patent Application No. 10-2022-0009779, filed on Jan. 24, 2022, at the Korean Intellectual Property Office, the entire disclosure of which is incorporated herein by reference for all purposes.

### BACKGROUND

### 1. Field

The following description relates to determining a molecular conformation, and more particularly, to determining a three-dimensional (3D) ground state conformation of a molecule.

### 2. Description of Related Art

Molecules in a natural state exist in a stable state, so molecular force acts in a direction in which the energy is weaker. When molecular information is given, an initial conformation is generated based on conditions for interatomic distance and angles and the like, and a final ground state conformation is determined by repeating a process of determining an energy value at the time an initial conformation is generated and a direction (gradient) in which energy weakens, and gradually modifying the initial conformation until convergence is achieved.

However, finding a ground state conformation using a gradient may involve long calculations and there may be a risk of reaching a local minimum and not a global minimum.

### SUMMARY

This Summary is provided to introduce a selection of concepts in a simplified form that are further described below in the Detailed Description. This Summary is not intended to identify key features or essential features of the claimed subject matter, and is not intended to be used as an aid in determining the scope of the claimed subject matter.

In one general aspect, there is provided a processor-implemented method of determining a molecular conformation, the method including generating candidate conformations based on one or more artificial neural network (ANN)-based conformation generative model that is based on an artificial neural network (ANN), comparing energy values between the candidate conformations by inputting the candidate conformations to an ANN-based conformation selecting model, and determining a final conformation based on a result of the comparing.

The determining of the final conformation may include determining a candidate conformation with a lowest energy value from among the candidate conformations as the final conformation.

The ANN-based conformation selecting model may be trained to determine a ranking of a difference between the energy value and a target energy value of each of the candidate conformations.

The comparing of the energy values may include comparing a predicted value of a loss function of each of the candidate conformations by inputting the candidate conformations to the ANN-based conformation selecting model.

The generating of the candidate conformations may include generating the candidate conformations corresponding to molecular information by inputting the molecular information to each of the one or more ANN-based conformation generative models.

The generating of the candidate conformations may include generating a number of candidate conformations corresponding to molecular information by inputting the molecular information a number of times to the one or more ANN-based conformation generative model.

The determining of the final conformation may include comparing a lowest predicted value of a loss function from among predicted values of loss functions of the candidate conformations with a threshold value, and determining a candidate conformation corresponding to a minimum difference between the lowest predicted value and the threshold value as the final conformation.

The method may include predicting the energy values of each of the candidate conformations by inputting the candidate conformations to an ANN-based energy prediction model.

The predicting of the energy values may include comparing the energy values by referring to each energy value of the candidate conformations predicted through the ANN-based energy prediction model.

In another general aspect, there is provided an apparatus for determining molecular conformation, the apparatus including a processor configured to generate a candidate conformations based on one or more artificial neural network (ANN)-based conformation generative model, to compare energy values between the candidate conformations by inputting the candidate conformations to an ANN-based conformation selecting model, and to determine a final conformation based on a result of the comparing.

The processor may be configured to determine a candidate conformation with a lowest energy value from among the candidate conformations as the final conformation.

The ANN-based conformation selecting model may be trained to determine a ranking of a difference between the energy value and a target energy value of each of the candidate conformations.

The processor may be configured to compare a predicted value of a loss function of each of the candidate conformations by inputting the candidate conformations to the ANN-based conformation selecting model.

The processor may be configured to generate candidate conformations corresponding to molecular information by inputting the molecular information to each of the one or more ANN-based conformation generative models.

The processor may be configured to generate a number of candidate conformations corresponding to molecular information by inputting the molecular information a number of times to the one or more ANN-based conformation generative model.

The processor may be configured to compare a lowest predicted value of a loss function from among predicted values of loss functions of the candidate conformations with a threshold value, and to determine a candidate conformation corresponding to a minimum difference between the lowest predicted value and the threshold value as the final conformation.

The processor may be configured to predict the energy values of each of the candidate conformations by inputting the candidate conformations to an ANN-based energy prediction model.

The processor may be configured to compare the energy values by referring to each energy value of the candidate conformations predicted through the ANN-based energy prediction model.

Other features and aspects will be apparent from the following detailed description, the drawings, and the claims.

### BRIEF DESCRIPTION OF THE DRAWINGS

FIG. 1 illustrates an example of a method of representing a molecule.

FIG. 2A illustrates an example of a deep learning operation method based on an artificial neural network (ANN).

FIG. 2B illustrates an example of a conformation determination system.

FIG. 3 illustrates an example of a method of determining a molecular conformation.

FIG. 4 illustrates an example of an operation of a conformation selecting model.

FIG. 5 illustrates an example of an operation of a conformation selecting apparatus to which an energy prediction model is added.

FIG. 6 illustrates an example of a configuration of an apparatus for determining a molecular conformation.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order.

The features described herein may be embodied in different forms and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

Although the terms first, second, third, A, B, C, (a), (b), (c), and the like are used to explain various components, the components are not limited by the terms. These terms should be used only to distinguish one component from another component. For example, a “first” component may be referred to as a “second” component, or similarly, a “second” component may be referred to as a “first” component.

It will be understood that when a component is referred to as being “connected to” or “coupled to” another component, the component may be directly connected or coupled to the other component or intervening components may be present. However, when a component is referred to as being “directly connected to” or “directly coupled to” another component, it will be understood that no intervening components are present. Terms that explain the relationship between components such as “between-” and “precisely between” or “adjacent to” and “directly adjacent to” will be understood likewise.

The terminology used herein is for the purpose of describing particular examples only and is not to be limiting of the examples. As used herein, the singular forms “a”, “an”, and “the” are intended to include the plural forms as well, unless the context clearly indicates otherwise. As used herein, the terms “include,” “comprise,” and “have” specify the presence of stated features, numbers, operations, elements, components, and/or combinations thereof, but do not preclude the presence or addition of one or more other features, numbers, operations, elements, components, and/or combinations thereof.

Unless otherwise defined, all terms used herein including technical or scientific terms have the same meaning as commonly understood by one of ordinary skill in the art. Terms, such as those defined in commonly used dictionaries, are to be interpreted as having a meaning that is consistent with the meaning they have in the context of the relevant art, and are not to be interpreted in an idealized or overly formal sense unless expressly so defined herein.

The use of the term “may” herein with respect to an example or embodiment (e.g., as to what an example or embodiment may include or implement) means that at least one example or embodiment exists where such a feature is included or implemented, while all examples are not limited thereto.

Examples may be implemented in various types of products such as personal computers (PCs), smart phones, laptop computers, tablet computers, smartphones, televisions (TVs), smart home appliances, intelligent vehicles, kiosks, wearable devices, and the like. Hereinafter, examples will be described in detail with reference to the accompanying drawings. When describing the examples with reference to the accompanying drawings, like reference numerals refer to like components.

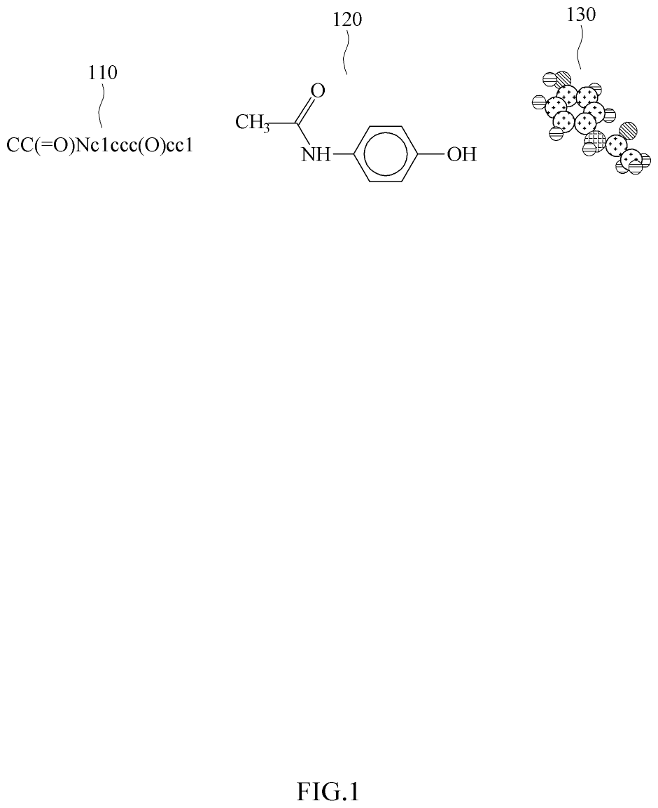

FIG. 1 illustrates an example of a method of representing a molecule.

A method of representing a molecule may be a one-dimensional (1D) simplified molecular-input line-entry system (SMILES) 110, a two-dimensional (2D) molecular graph 120, a three-dimensional (3D) conformation 130, and the like. Among these methods, the 3D conformation 130, which is 3D location information of a molecule, may be the most natural way to represent a molecule without loss of information.

A molecule may have several spatial configurations, and as a size of a molecule increases, a degree of freedom of a molecule increases, and accordingly, a molecule may have various conformations. A most stable state among conformations of a molecule is referred to as a ground state conformation, in which energy of the molecule may be minimized. Values of physical properties in a ground state may be an important indicator of a substance and may provide information for understanding molecules.

When molecular information is given, an initial conformation may be generated based on conditions for interatomic distance, angles and the like, and a final ground state conformation may be determined by repeating a process of determining an energy value at the time an initial conformation is generated and a direction (gradient) in which energy weakens, and gradually modifying the initial conformation until convergence. In addition, recently, models that may generate conformations when a molecular graph is given as an input using machine learning are being developed.

Finding a ground state conformation is generally known to be non-deterministic polynomial-time (NP)-hard. For example, it may be more difficult to find a ground state conformation when a size of a molecule is large because the molecule can have various and complex conformations due to a high degree of freedom (i.e., rotatable bonds).

In particular, if a ground state conformation is found by using a gradient, calculation may take too long or there may be a risk of reaching a local minimum and not a global minimum.

Further, conformation generation methods in machine learning according to a related art are models trained to learn a probability distribution for all conformations that a given molecule may have and there may not be a specialized method for obtaining a ground state conformation.

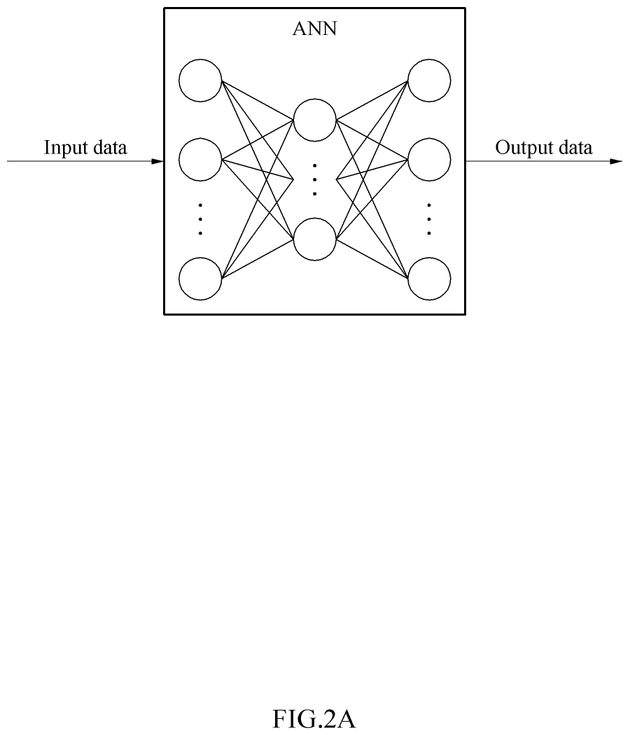

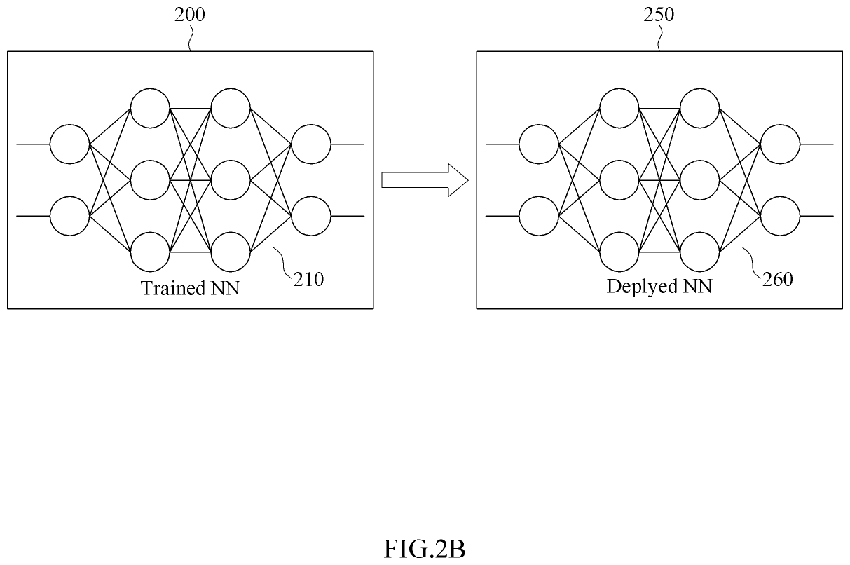

Accordingly, an example of a method of determining a molecular conformation may directly generate ground state conformations based on a conformation generative model that is based on an artificial neural network (ANN) without optimizing conformationswill be described below. However, since generation of the conformation generative model that is based on an ANN is based on a probability distribution, the conformation generative model may have some degree of randomness even if the ground state conformations are learned and are a target during generation. This is because actual ground state conformations are a deterministic finite number of conformations and are not determined probabilistically. To compensate for this, a conformation selecting model that is based on machine learning may be additionally used in the method of determining a molecular conformation. Before operations of the conformation generative model and the conformation selecting model that are based on an ANN are described, the ANN will be described with reference to FIGS. 2A and 2B.

FIG. 2A illustrates an example of a deep learning operation method based on an ANN.

The neural network or ANN may generate mapping between input information and output information, and may have a generalization capability to infer a relatively correct output with respect to input information that has not been used for training. The neural network may refer to a general model that has an ability to solve a problem or perform tasks, as non-limiting examples, where nodes form the network through connections and other parameter adjustment through training.

The neural network may be implemented as an architecture having a plurality of layers including an input layer, hidden information and layers, and an output. In the neural network layer, an input data or map may be convoluted with a filter called a kernel, and as a result, a plurality of feature maps may be output. The output feature maps may be again convoluted in a subsequent convolutional layer as input feature maps with another kernel, and a plurality of new feature maps may be output. After the convolution operations are repeatedly performed, and potentially, other layer operations performed, the recognition or classification results of features of the input data through the neural network may be finally output, as output data in a non-limiting examples.

The ANN may be a machine learning model structure. In another example, a neural network layer may extract feature data from input data and provide an inference based on the feature data. The feature data may also be data associated with a feature obtained by abstracting input data. The neural network may map input data and output data in a nonlinear relationship based on deep learning, to generate such inferences. Deep learning, such as, through back propagation for multiple hidden layers of a neural network may generate a trained neural network for various purposes or tasks, may map input data and output data to each other through supervised and/or unsupervised learning, as only examples.

In an example, training an artificial neural network may indicate determining and adjusting weights and biases between layers or weights and biases among a plurality of nodes belonging to different layers adjacent to one another, as only non-limiting examples of such parameters.

In an example, the neural network may be, for example, a deep neural network (DNN), a convolutional neural network (CNN), a recurrent neural network (RNN), a restricted Boltzmann machine (RBM), a deep belief network (DBN), a bidirectional recurrent DNN (BRDNN), a deep Q-network, or a combination of two or more thereof, but examples thereof are not limited to the foregoing examples. The neural network may include a hardware structure that may be implemented through execution of instructions by a processor

FIG. 2A illustrates a structure of the ANN (e.g., a CNN) that receives input data and outputs output data. The ANN may be, for example, a deep neural network (DNN) including at least two layers.

FIG. 2B illustrates an example of a conformation determination system.

Referring to FIG. 2B, the conformation determination system may include a training apparatus 200 and an apparatus for determining a molecular conformation 250. The training apparatus 200 may correspond to a computing device with various processing functions such as generating a neural network, training (or learning) the neural network, or retraining the neural network. For example, the training apparatus 200 may be implemented with various types of devices such as personal computers (PCs), server devices, mobile devices, and the like.

The training apparatus 200 may generate one or more trained neural network 210 by repeatedly training (learning) a given initial neural network. Generating the one or more trained neural network 210 may be determining parameters of a neural network. Here, the parameters may include various types of data input and/or output to and/or from the neural network such as input/output activations, weights, biases, and the like of the neural network. As repetitive training of the neural network proceeds, parameters of the neural network may be tuned to compute a more accurate output for a given input.

The training apparatus 200 may transmit the one or more trained neural network 210 to the apparatus for determining a molecular conformation 250. The apparatus for determining a molecular conformation 250 may be included in a mobile device, an embedded device, and the like. The apparatus for determining a molecular conformation 250 may be dedicated hardware for driving the neural network.

The apparatus for determining a molecular conformation 250 may drive the one or more trained neural network 210 without a change, or the one or more trained neural network 210 may drive a processed (e.g., quantized) neural network 260. The apparatus for determining a molecular conformation 250 that drives the processed neural network 260 may be implemented in an independent device separate from the training apparatus 200. However, the examples are not limited thereto, and in an example, the apparatus for determining a molecular conformation 250 may also be implemented in the same device as the training apparatus 200.

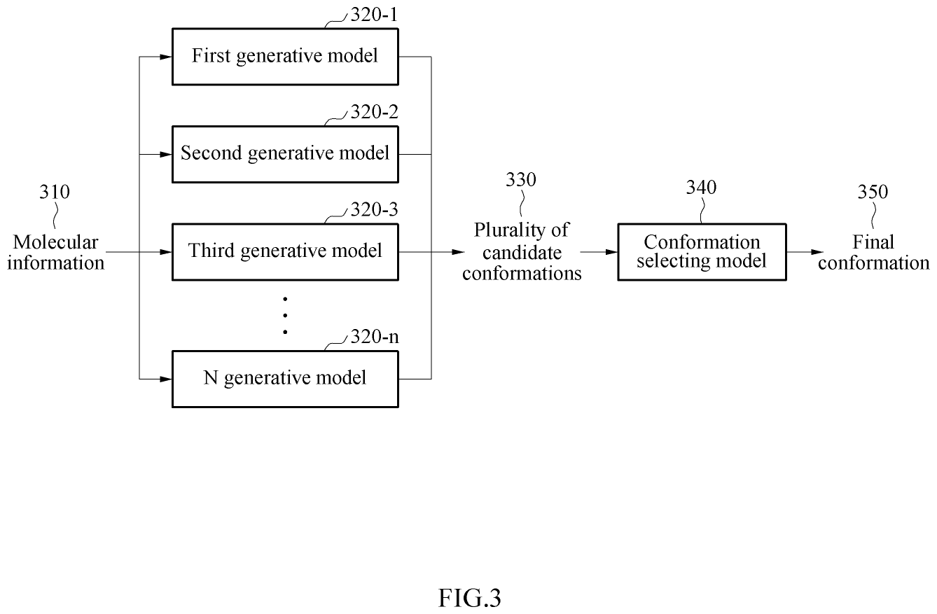

FIG. 3 illustrates an example of a method of determining a molecular conformation.

A plurality of candidate conformations 330 corresponding to molecular information 310 may be generated by inputting the molecular information 310 to one or more conformation generative model 320-1 through 320-n that are based on an ANN.

The conformation generative models 320-1 through 320-n that are based on an ANN may be a model that generates a conformation when molecular information (i.e., a molecular graph) is given as an input, and may be trained based on ground state conformation data.

For example, the conformation generative models 320-1 through 320-n that are based on an ANN may be a conditional graph continuous flow (CGCF) model that generates a molecular conformation based on a continuous normalizing flow and energy tilting. The continuous normalizing flow may be a generative machine learning technique by which a desired probability distribution through inverse functions from a known or simple probability distribution is learned. The energy tilting may be a scheme that improves a generative model by using an energy function that is an unnormalized probability function. The CGCF model may use these techniques to generate an interatomic distance of a molecule and calculate a conformation therefrom.

In another example, the conformation generative models 320-1 through 320-n that are based on an ANN may include a ConfGF model using a score function that is a differential value of a log-likelihood function. When a molecule is given, the ConfGF model may find a scores function value of a probability distribution for an interatomic distance and generates a conformation by using the score function value like a force field.

The conformation generative models 320-1 through 320-n that are based on an ANN may not need to perform an optimization through calculation of gradients by directly learning and generating ground state conformations.

In addition, since an optimum model for a molecular type and structure may be different, the conformation generative models 320-1 through 320-n that are based on an ANN may generate various, pluralities of candidate conformations 330 in or near a ground state by using as many models and/or parameters as possible. The plurality of candidate conformations 330 to be generated are generated with a ground state conformation as a target but may be affected by a randomness of a generative model or a compatibility between a molecule and a model.

Accordingly, a conformation selecting model 340 may improve performance by selecting an optimum final conformation 350 among the plurality of candidate conformations 330. Generating a plurality of conformations and selecting the optimum final conformation 350 may help improve performance more, compared to directly generating conformations. For example, from a result of an experiment, it can be confirmed that an average energy difference of “38” organic light emitting diodes (OLED) dopant molecules reduces to “2.2020”, “1.3672”, and “0.98481” when the number of generated conformations increases.

When the optimum final conformation 350 is to be selected among a plurality of conformations, a conformation in which energy is weakest (i.e., a most stable state) in terms of molecular energy, may be selected. Here, to find a ground state conformation based on a molecular structure according to conventional processes, a density functional theory (DFT) calculation may be performed, or an energy function provided by an RDKit may be used, but the DFT calculation may take a long time and the RDKit may be inaccurate.

Accordingly, a method of determining a molecular conformation may use a conformation selecting model 340 that is based on an ANN. The conformation selecting model 340 may compare energy values between a plurality of candidate conformations 330 by inputting the plurality of candidate conformations 330 to a conformation selecting model that is based on an ANN. Further, the conformation selecting model 340 may determine any one of the conformations among the plurality of candidate conformations 330 as a final conformation 350 based on a result of the comparing.

The conformation selecting model 340 may determine a candidate conformation with a lowest energy value (i.e., a ground state) among the plurality of candidate conformations 330 as the final conformation 350.

More specifically, information needed to select a conformation may be sufficient if conformations can be compared to determine which conformation has the lowest energy value, not an exact energy value. Accordingly, the conformation selecting model 340 may determine the final conformation 350 based on only a ranking of energy values between the plurality of candidate conformations 330 without finding the energy values of the plurality of candidate conformations 330.

The aim of the conformation selecting model 340 may be to select a final conformation 350 that has energy closest to a ground state energy among the plurality of candidate conformations 330 that are generated, which may be expressed as an equation represented by Equation 1 below.

$$arg {min}_{i} ( \hat{E} ( {R}_{i} ) - \hat{E} ( {R}^{+} ) ) \qquad [ Equation 1 ]$$

In Equation 1, Ê(Rᵢ) may denote an actual energy value of conformation, Rᵢ, R⁺ may denote a ground state conformation, Ê(R⁺) may denote a target energy value, R₁, . . . , Rₙ may denote a plurality of candidate conformations 330, and Rᵢ may denote a final conformation 350.

That is, the conformation selecting model 340 may determine a conformation that minimizes a loss function î(Rᵢ)=Ê(Rᵢ)−Ê(R⁺) as the final conformation 350. To calculate the loss function \hat{l}, a DFT calculation may be performed so the calculation may take a long time. Accordingly, the conformation selecting model 340 may use a loss function prediction model l that is based on machine learning instead of performing a DFT calculation, which may be costly.

When the loss function prediction model l is trained, a loss function Lₗₒₛₛ(\hat{l}ᵢⱼ, \hat{l}ᵢⱼ) (loss-prediction loss) may be set for each conformation ordered pair (Rᵢ, Rⱼ) as shown by Equation 2 below.

$${L}_{loss} ( {\hat{l}}_{ij} , {l}_{ij} ) = max ( 0 , - 𝕀 ( {l}_{i} , {l}_{j} ) \cdot ( {\hat{l}}_{i} - {\hat{l}}_{j} ) + ξ \qquad [ Equation 2 ]$$

$$𝕀 ( {l}_{i} , {l}_{j} ) = \{ \begin{matrix}1 , & {l}_{i} > {l}_{j} \\ - 1 , & otherwise\end{matrix} , ξ : margin$$

Unlike general examples in which a mean absolute error is used, Lₗₒₛₛ may be trained to distinguish rankings between lᵢ=l(Rᵢ). For example, with respect to a target loss function, if \hat{l}ᵢ>\hat{l}ⱼ and \hat{l}ᵢ>\hat{l}ⱼ, Lₗₒₛₛ is “0”, otherwise Lₗₒₛₛ is a positive value. Therefore, if training is sufficiently performed, if \hat{l}(R₁)<\hat{l}(R₂)< . . . <\hat{l}(Rₙ), then l(R₁)<l(R₂)< . . . <l(Rₙ) is established with respect to a generated conformation Rᵢ.

That is, if the conformation selecting model 340 is trained based on Lₗₒₛₛ, the conformation selecting model 340 may be trained to learn a ranking of energy values. When a training is completed, the conformation selecting model 340 may determine the ranking of energy values between the plurality of candidate conformations 330 and determine a candidate conformation with a lowest energy value as the final conformation 350. Here, if a value of \hat{l}(Rᵢ) is a minimum value, a value of Ê(Rᵢ)−Ê(R⁺) may be a minimum value.

A process of generating and selecting conformations may be repeated as many times as desired, but a condition under which iteration stops may also be set. A loss function l is a difference from a target energy, so if the process is set to be repeated until a value of l is below a threshold value, as many conformations close to a target as desired may be generated.

In an example, the conformation selecting model 340 may compare a lowest predicted value of a loss function among predicted values of loss functions of a plurality of candidate conformations with a threshold value, and determine a candidate conformation corresponding to a first lowest predicted value of a loss function less than the threshold value as the final conformation.

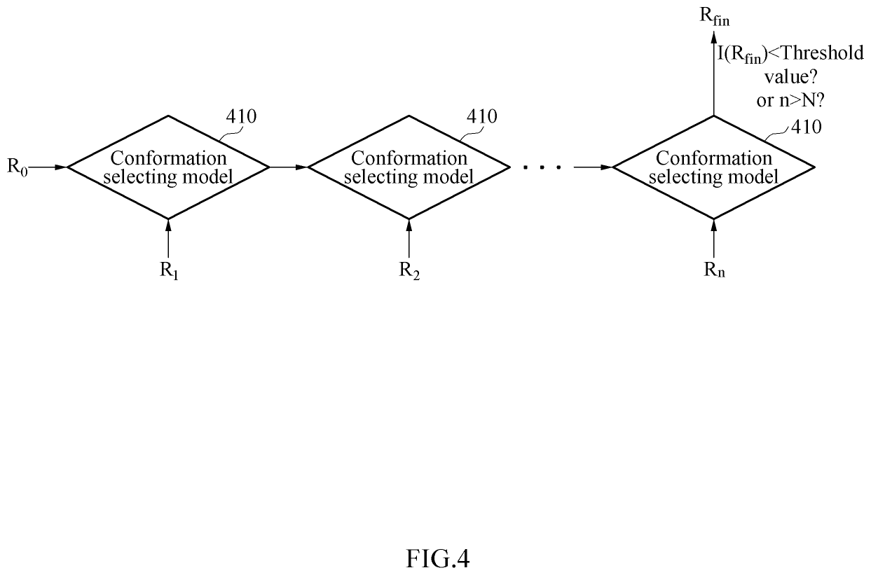

FIG. 4 illustrates an example of an operation of a conformation selecting model.

Referring to FIG. 4, a conformation selecting model 410 may be the conformation selecting model 340 described with reference to FIG. 3. The description of FIG. 3 may also apply to FIG. 4, and accordingly, redundant description is not repeated herein.

The conformation selecting model 410 may compare energy values between a plurality of candidate conformations. For example, the conformation selecting model 410 may determine a candidate conformation with a lower energy value between two candidate conformations R₀ and R₁ that are generated from a conformation generative model. In addition, the conformation selecting model 410 may compare a candidate conformation that is determined to have a lower energy value from among the candidate conformations R₀ and R₁ with an energy value of R₃. The conformation selecting model 410 may perform a comparison until a condition is satisfied to determine a final conformation.

When the conformation selecting model 410 is used, a sufficiently accurate conformation may be obtained only by comparing energy between conformations, even if an energy value of a molecule is not accurately obtained.

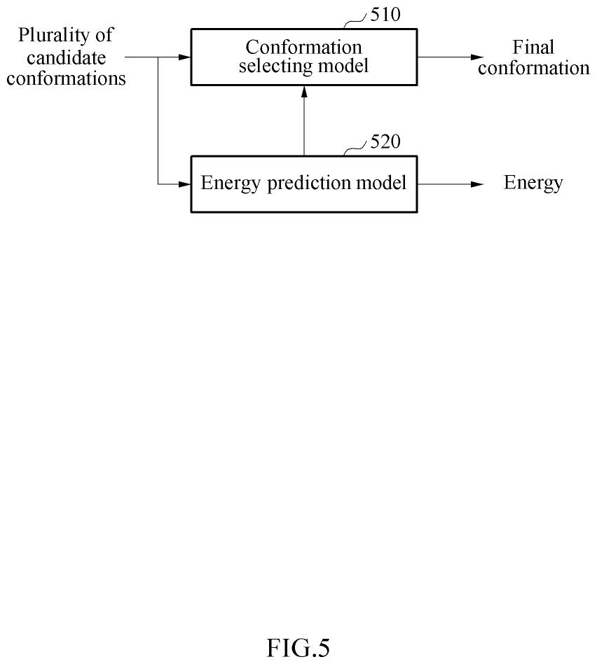

FIG. 5 illustrates an example of an operation of a conformation selecting system to which an energy prediction model is added.

FIGS. 3 and 4 illustrate a model for predicting a ranking of a difference (a target loss) between a ground state energy and energy of a conformation that is generated, without actually predicting the energy. Referring to FIG. 5, in an example, an energy prediction model 520 may be added to the conformation selecting system, in addition to a conformation selecting model 510.

Prediction of energy and conformation rankings may be simultaneously performed by inputting an intermediate value of the energy prediction model 520 to a loss function prediction model. The energy prediction model 520 that is based on an ANN may receive a plurality of candidate conformations as input and predict an energy value of each of the plurality of candidate conformations.

Further, the conformation selecting model 510 may compare energy values by considering the energy value of each of the plurality of candidate conformations that are predicted through the energy prediction model.

In an example, when the conformation selecting model 510 is trained, a loss (E−Ê)² or |E−Ê| for an energy prediction function and a loss Lₗₒₛₛ for a loss-prediction function may be used together. Intermediate calculated values of the energy prediction model 520 may be input to the conformation selecting model 510.

In an inference operation, energy prediction and conformation selection may be performed with an energy prediction model 520 that has been fully trained and a loss-prediction function l. When a conformation Rᵢ generated in each operation is input, an energy prediction value E(Rᵢ) is calculated and output, and the conformation selecting model 510 may compare an l value with an existing conformation and select a conformation with a lesser value.

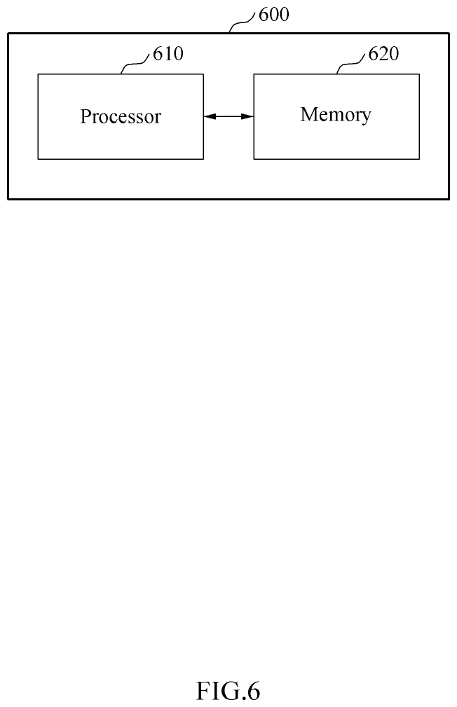

FIG. 6 illustrates an example of an apparatus for determining a molecular conformation.

Referring to FIG. 6, an apparatus for determining a molecular conformation 600 may be configured to determine a conformation of a predetermined state (i.e., a ground state) of a molecule. The apparatus for determining a molecular conformation 600 may include one or more processor 610 and a memory 620.

The memory 620 may store a variety of data used by components (e.g., the processor 610) of the apparatus for determining a molecular conformation 600. A variety of data may include, for example, computer-readable instructions and input data or output data for an operations related thereto. The memory 620 may include any one or any combination of a volatile memory and a non-volatile memory. The volatile memory device may be implemented as a dynamic random-access memory (DRAM), a static random-access memory (SRAM), a thyristor RAM (T-RAM), a zero capacitor RAM (Z-RAM), or a twin transistor RAM (TTRAM).

The non-volatile memory device may be implemented as an electrically erasable programmable read-only memory (EEPROM), a flash memory, a magnetic RAM (MRAM), a spin-transfer torque (STT)-MRAM, a conductive bridging RAM(CBRAM), a ferroelectric RAM (FeRAM), a phase change RAM (PRAM), a resistive RAM (RRAM), a nanotube RRAM, a polymer RAM (PoRAM), a nano floating gate Memory (NFGM), a holographic memory, a molecular electronic memory device), or an insulator resistance change memory. Further details regarding the memory 620 is provided below.

The memory 620 may store a generative model that is based on a pre-trained ANN.

The processor 610 may control an overall operation of the computing apparatus 200 and may execute corresponding processor-readable instructions for performing operations of the apparatus for determining a molecular conformation 600. The processor 610 may execute, for example, software, to control one or more hardware components of the apparatus for determining a molecular conformation 600 connected to the processor 610 and may perform various data processing or operations, and control of such components.

In an example, as at least a part of data processing or operations, the processor 610 may store instructions or data in the memory 620, execute the instructions and/or process data stored in the memory 620, and store resulting data obtained therefrom in the memory 620. The processor 200 may be a data processing device implemented by hardware including a circuit having a physical structure to perform desired operations. For example, the desired operations may include code or instructions included in a program.

The hardware-implemented device may include, for example, a microprocessor, a central processing unit (CPU), a graphic processing unit (GPU), a processor core, a multi-core processor, a multiprocessor, an application-specific integrated circuit (ASIC), a field programmable gate array (FPGA), an application processor (AP), a neural processing unit (NPU), an image signal processor (ISP), a sensor hub processor, or a communication processor (CP)and the like. Further details regarding the processor 210 is provided below.

The processor 610 may control the apparatus for determining a molecular conformation 600 to execute any one or any combination of the operations and functions described above with reference to FIGS. 2A through 6.

The processor 610 may control the apparatus for determining a molecular conformation 600 to generate a plurality of candidate conformations based on one or more conformation generative model that is based on an ANN, compare energy values between the plurality of candidate conformations by inputting the plurality of candidate conformations to a conformation selecting model that is based on an ANN, and determine a final conformation based on a result of the comparing.

The processor 610 may read/write neural network data, for example, input data, feature map data, kernel data, biases, weights, for example, connection weight data, hyperparameters, and other parameters etc., from/to the memory 620 and implement a neural network using the read/written data. When the neural network is implemented, the processor 610 may repeatedly perform operations between an input and parameters, in order to generate data with respect to an output. Here, in an example convolution layer, a number of convolution operations may be determined, depending on various factors, such as, for example, the number of channels of the input or input feature map, the number of channels of the kernel, a size of the input feature map, a size of the kernel, number of the kernels, and precision of values. Such a neural network may be implemented as a complicated architecture, where the processor 610 performs convolution operations with an operation count of up to hundreds of millions to tens of billions, and the frequency at which the processor 610 accesses the memory 620 for the convolution operations rapidly increases.

The training apparatus 200, the apparatus for determining a molecular conformation 250, the apparatus for determining a molecular conformation 600, and other apparatuses, devices, units, modules, and components described herein are implemented by hardware components. Examples of hardware components that may be used to perform the operations described in this application where appropriate include controllers, sensors, generators, drivers, memories, comparators, arithmetic logic units, adders, subtractors, multipliers, dividers, integrators, and any other electronic components configured to perform the operations described in this application. In other examples, one or more of the hardware components that perform the operations described in this application are implemented by computing hardware, for example, by one or more processors or computers. A processor or computer may be implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices that is configured to respond to and execute instructions in a defined manner to achieve a desired result. In one example, a processor or computer includes, or is connected to, one or more memories storing instructions or software that are executed by the processor or computer. Hardware components implemented by a processor or computer may execute instructions or software, such as an operating system (OS) and one or more software applications that run on the OS, to perform the operations described in this application. The hardware components may also access, manipulate, process, create, and store data in response to execution of the instructions or software. For simplicity, the singular term “processor” or “computer” may be used in the description of the examples described in this application, but in other examples multiple processors or computers may be used, or a processor or computer may include multiple processing elements, or multiple types of processing elements, or both. For example, a single hardware component or two or more hardware components may be implemented by a single processor, or two or more processors, or a processor and a controller. One or more hardware components may be implemented by one or more processors, or a processor and a controller, and one or more other hardware components may be implemented by one or more other processors, or another processor and another controller. One or more processors, or a processor and a controller, may implement a single hardware component, or two or more hardware components. A hardware component may have any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, multiple-instruction multiple-data (MIMD) multiprocessing, a controller and an arithmetic logic unit (ALU), a DSP, a microcomputer, an application-specific integrated circuit (ASIC), a field programmable gate array (FPGA), a programmable logic unit (PLU), a central processing unit (CPU), a graphics processing unit (GPU), a neural processing unit (NPU), or any other device capable of responding to and executing instructions in a defined manner.

The methods that perform the operations described in this application are performed by computing hardware, for example, by one or more processors or computers, implemented as described above executing instructions or software to perform the operations described in this application that are performed by the methods. For example, a single operation or two or more operations may be performed by a single processor, or two or more processors, or a processor and a controller. One or more operations may be performed by one or more processors, or a processor and a controller, and one or more other operations may be performed by one or more other processors, or another processor and another controller. One or more processors, or a processor and a controller, may perform a single operation, or two or more operations.

The Instructions or software to control a processor or computer to implement the hardware components and perform the methods as described above are written as computer programs, code segments, instructions or any combination thereof, for individually or collectively instructing or configuring the processor or computer to operate as a machine or special-purpose computer to perform the operations performed by the hardware components and the methods as described above. In one example, the instructions or software include machine code that is directly executed by the processor or computer, such as machine code produced by a compiler. In an example, the instructions or software includes at least one of an applet, a dynamic link library (DLL), middleware, firmware, a device driver, an application program storing the method of determining a molecular conformation. In another example, the instructions or software include higher-level code that is executed by the processor or computer using an interpreter. Programmers of ordinary skill in the art can readily write the instructions or software based on the block diagrams and the flow charts illustrated in the drawings and the corresponding descriptions in the specification, which disclose algorithms for performing the operations performed by the hardware components and the methods as described above.

The instructions or software to control a processor or computer to implement the hardware components and perform the methods as described above, and any associated data, data files, and data structures, are recorded, stored, or fixed in or on one or more non-transitory computer-readable storage media. Examples of a non-transitory computer-readable storage medium include read-only memory (ROM), random-access programmable read only memory (PROM), electrically erasable programmable read-only memory (EEPROM), random-access memory (RAM), magnetic RAM (MRAM), spin-transfer torque(STT)-MRAM, static random-access memory (SRAM), thyristor RAM (T-RAM), zero capacitor RAM (Z-RAM), twin transistor RAM (TTRAM), conductive bridging RAM(CBRAM), ferroelectric RAM (FeRAM), phase change RAM (PRAM), resistive RAM(RRAM), nanotube RRAM, polymer RAM (PoRAM), nano floating gate Memory(NFGM), holographic memory, molecular electronic memory device), insulator resistance change memory, dynamic random access memory (DRAM), static random access memory (SRAM), flash memory, non-volatile memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, blue-ray or optical disk storage, hard disk drive (HDD), solid state drive (SSD), flash memory, a card type memory such as multimedia card micro or a card (for example, secure digital (SD) or extreme digital (XD)), magnetic tapes, floppy disks, magneto-optical data storage devices, optical data storage devices, hard disks, solid-state disks, and any other device that is configured to store the instructions or software and any associated data, data files, and data structures in a non-transitory manner and providing the instructions or software and any associated data, data files, and data structures to a processor or computer so that the processor or computer can execute the instructions. In an example, the instructions or software and any associated data, data files, and data structures are distributed over network-coupled computer systems so that the instructions and software and any associated data, data files, and data structures are stored, accessed, and executed in a distributed fashion by the one or more processors or computers

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. .

Therefore, the scope of the disclosure is defined not by the detailed description, but by the claims and their equivalents, and all variations within the scope of the claims and their equivalents are to be construed as being included in the disclosure.

What is claimed is:

1. A processor-implemented method of determining a molecular conformation, the method comprising:
generating candidate conformations based on one or more artificial neural network (ANN)-based conformation generative model;
comparing energy values between the candidate conformations by inputting the candidate conformations to an ANN-based conformation selecting model; and
determining a final conformation based on a result of the comparing.

2. The method of claim 1, wherein the determining of the final conformation comprises determining a candidate conformation with a lowest energy value from among the candidate conformations as the final conformation.

3. The method of claim 1, wherein the ANN-based conformation selecting model is trained to determine a ranking of a difference between the energy value and a target energy value of each of the candidate conformations.

4. The method of claim 1, wherein the comparing of the energy values comprises comparing a predicted value of a loss function of each of the candidate conformations by inputting the candidate conformations to the ANN-based conformation selecting model.

5. The method of claim 1, wherein the generating of the candidate conformations comprises generating the candidate conformations corresponding to molecular information by inputting the molecular information to each of the one or more ANN-based conformation generative models.

6. The method of claim 1, wherein the generating of the candidate conformations comprises generating a number of candidate conformations corresponding to molecular information by inputting the molecular information a number of times to the one or more ANN-based conformation generative model.

7. The method of claim 1, wherein the determining of the final conformation comprises:
comparing a lowest predicted value of a loss function from among predicted values of loss functions of the candidate conformations with a threshold value; and
determining a candidate conformation corresponding to a minimum difference between the lowest predicted value and the threshold value as the final conformation.

8. The method of claim 1, further comprising:
predicting the energy values of each of the candidate conformations by inputting the candidate conformations to an ANN-based energy prediction model.

9. The method of claim 8, wherein the predicting of the energy values comprises comparing the energy values by referring to each energy value of the candidate conformations predicted through the ANN-based energy prediction model.

10. A non-transitory computer-readable storage medium storing instructions that, when executed by a processor, cause the processor to perform the method of claim 1.

11. An apparatus for determining molecular conformation, the apparatus comprising:
a processor configured to generate a candidate conformations based on one or more artificial neural network (ANN)-based conformation generative model, to compare energy values between the candidate conformations by inputting the candidate conformations to an ANN-based conformation selecting model, and to determine a final conformation based on a result of the comparing.

12. The apparatus of claim 11, wherein the processor is further configured to determine a candidate conformation with a lowest energy value from among the candidate conformations as the final conformation.

13. The apparatus of claim 11, wherein the ANN-based conformation selecting model is trained to determine a ranking of a difference between the energy value and a target energy value of each of the candidate conformations.

14. The apparatus of claim 11, wherein the processor is further configured to compare a predicted value of a loss function of each of the candidate conformations by inputting the candidate conformations to the ANN-based conformation selecting model.

15. The apparatus of claim 11, wherein the processor is further configured to generate candidate conformations corresponding to molecular information by inputting the molecular information to each of the one or more ANN-based conformation generative models.

16. The apparatus of claim 11, wherein the processor is further configured to generate a number of candidate conformations corresponding to molecular information by inputting the molecular information a number of times to the one or more ANN-based conformation generative model.

17. The apparatus of claim 11, wherein the processor is further configured to compare a lowest predicted value of a loss function from among predicted values of loss functions of the candidate conformations with a threshold value, and to determine a candidate conformation corresponding to a minimum difference between the lowest predicted value and the threshold value as the final conformation.

18. The apparatus of claim 11, wherein the processor is further configured to predict the energy values of each of the candidate conformations by inputting the candidate conformations to an ANN-based energy prediction model.

19. The apparatus of claim 18, wherein the processor is further configured to compare the energy values by referring to each energy value of the candidate conformations predicted through the ANN-based energy prediction model.
